(19) United States
(12) Patent Application Publication
(10) Pub. No.: US 2017/0167985 A1
(43) Pub. Date: Jun. 15, 2017

(54) SPECIMEN ANALYSIS APPARATUS AND METHOD OF ANALYZING SPECIMEN USING THE SAME

(71) Applicant: Samsung Electronics Co., Ltd, Gyeonggi-do (KR)

(72) Inventors: Beom Seok Lee, Gyeonggi-do (KR); Sung Ha Park, Gyeonggi-do (KR); Kyung-Mi Song, Gyeonggi-do (KR); Sang Hyun Lee, Gyeonggi-do (KR); Chung Ung Kim, Gyeonggi-do (KR)

(21) Appl. No.: 15/374,859

(22) Filed: Dec. 9, 2016

(30) Foreign Application Priority Data
Dec. 10, 2015 (KR) 10-2015-0176087

(51) Int. Cl. G01N 21/80; G01N 33/487; G01N 33/84

(52) U.S. Cl. CPC G01N 21/80; G01N 33/84; G01N 33/48785

(57) ABSTRACT

Disclosed herein are specimen analysis apparatus and method of analyzing specimen using the same. The specimen analysis apparatus includes a cartridge configured to accommodate at least three pH indicators having different properties and an analyzer configured to determine whether a specimen is acidic or basic using one of the pH indicators. The analyzer determines a pH measurement value using another pH indicator selected based on whether the specimen is acidic or basic.

### CROSS-REFERENCE TO RELATED APPLICATION(S) AND CLAIM OF PRIORITY

The present application is related to and claims the benefit of Korean Patent Application No. 10-2015-0176087, filed on Dec. 10, 2015 in the Korean Intellectual Property Office, the disclosure of which is incorporated herein by reference.

### TECHNICAL FIELD

Embodiments of the present disclosure relate to specimen analysis apparatuses, and methods of analyzing specimens by using the specimen analysis apparatuses.

### BACKGROUND

Apparatuses and methods of analyzing specimen samples are required in various fields such as environmental monitoring, food inspection, and medical diagnosis. Conventionally, in order to perform a test according to predetermined protocols, skilled experimenters manually perform various stages of the test such as repeated operations of injecting reagents, mixing, separating, moving, reacting, and centrifuging. These repeated operations may cause errors of test results.

Thus, compact and automated analysis apparatuses have been developed to quickly analyze a specimen. Particularly, since a specimen sample is quickly analyzed regardless locations by using a portable cartridge, structures and functions of the portable cartridge have been improved for use in various other fields. Recently, research has been conducted to develop methods of precisely analyzing specimen samples using compact and automated specimen analysis apparatuses.

### SUMMARY

To address the above-discussed deficiencies, it is a primary object to provide a specimen analysis apparatus includes a cartridge configured to accommodate at least three pH indicators having different properties; and an analyzer configured to judge whether a specimen is acidic or basic using one of the pH indicators, and determine one pH measurement value obtained using another pH indicator selected based on results of the judgement as a pH measurement value of the specimen.

Here, the cartridge accommodates at least three pH indicators having different color change ranges.

Also, the analyzer judges whether the specimen is acidic or basic using one pH indicator having a widest color change range among the at least three pH indicators, and determines one pH measurement value obtained using another pH indicator selected from at least two pH indicators having different color change ranges based on results of the judgement as the pH measurement value of the specimen.

Also, the specimen analysis apparatus further includes a controller configured to realize a user interface configured to provide analysis information about a target analyte in the specimen and information about whether a pH measurement value of the target analyte in the specimen is in a normal range and display the user interface on a display.

Also, the cartridge comprises a universal pH indicator, a basic pH indicator, and an acidic pH indicator.

Also, the analyzer measures a pH value using optical density.

Also, the cartridge comprises at least three pH indicators in the forms of at least one a solidified gel and a thin film.

In accordance with another aspect of the present disclosure, a specimen analysis apparatus includes an analyzer configured to deduce analysis information about a specimen and a pH measurement value of the specimen and analyze whether the pH measurement value is within a normal range; and a display configured to display a user interface configured to provide the analysis information about the specimen and analysis information about whether the pH measurement value is within the normal range.

Here, the display displays a user interface through which a user sets normal ranges of target analytes in the specimen.

Also, the display displays a user interface configured to set whether analysis information about whether the pH measure value is within the normal range

Also, the analyzer judges whether the specimen is acidic or basic using one pH indicator having a widest color change range among at least three pH indicators having different color change ranges and accommodated in a cartridge, and determines one pH measurement value obtained using another pH indicator selected based on results of the judgement as a pH measurement value of the specimen.

Also, the specimen analysis apparatus further includes a memory configured to store data about normal ranges of target analytes in the specimen.

In accordance with another aspect of the present disclosure, method of analyzing a specimen using a specimen analysis apparatus includes accommodating at least three pH indicators having different properties; judging whether a specimen is acidic or basic using one of the pH indicators; and determining a pH measurement value obtained using another pH indicator selected based on results of the judgement as a pH measurement value of the specimen.

Here, the accommodating is performed by accommodating at least three pH indicators having different color change ranges

Also, the determining is performed by judging whether the specimen is acidic or basic using one pH indicator having a widest color change range among the at least three pH indicators, and determining one pH measurement value obtained using another pH indicator selected from at least two pH indicators having different color change ranges based on results of the judgement as the pH measurement value of the specimen

In accordance with another aspect of the present disclosure, method of analyzing a specimen using a specimen analysis apparatus includes measuring a concentration of a target analyte in a specimen and a pH measurement value of a specimen; analyzing whether the pH measurement value is within a normal range; and displaying a user interface on a display realized to provide analysis information about the target analyte in the specimen and information about whether a pH measurement value of the target analyte in the specimen.

Here, the displaying includes displaying a user interface through which a user sets normal ranges of target analytes in the specimen.

Also, the displaying includes displaying a user interface configured to set whether analysis information about whether the pH measure value is within the normal range.

Before undertaking the DETAILED DESCRIPTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely. Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 illustrates a specimen analysis apparatus to which a cartridge is coupled according to an embodiment.

FIG. 2 illustrates an exploded view of the cartridge and a mounting member of the specimen analysis apparatus to which the cartridge is coupled according to an embodiment.

FIG. 3 illustrates a state in which the cartridge is coupled to the mounting member of the specimen analysis apparatus according to an embodiment.

FIG. 4 illustrates a cartridge according to an embodiment.

FIG. 5 illustrates an exploded view of a tester of the cartridge according to an embodiment.

FIG. 6 illustrates a cross-sectional view of the tester of the cartridge illustrated in FIG. 4 taken along line AA′.

FIG. 7 illustrates a control block diagram of a specimen analysis apparatus according to an embodiment.

FIGS. 8A and 8B illustrate graphs for describing activities of enzymes with respect to pH.

FIG. 9 illustrates a cartridge accommodating reagents having different properties in a plurality of wells according to an embodiment.

FIG. 10 illustrates a graph for describing color change ranges of pH indicators having different properties according to an embodiment.

FIG. 11A, FIG. 11B, FIG. 11C, FIG. 11D, FIG. 11E, and FIG. 11F illustrate user interface screens displayed on a display according to an embodiment.

FIG. 12 illustrates a flowchart for describing a process of measuring the pH of the specimen using the plurality of pH indicators by the specimen analysis apparatus according to an embodiment.

FIG. 13 illustrates a flowchart for describing a process of displaying a user interface configured to provide analysis information about the target analyte in the specimen together with analysis information about the pH value of the target analyte on the display by the specimen analysis apparatus.

### DETAILED DESCRIPTION

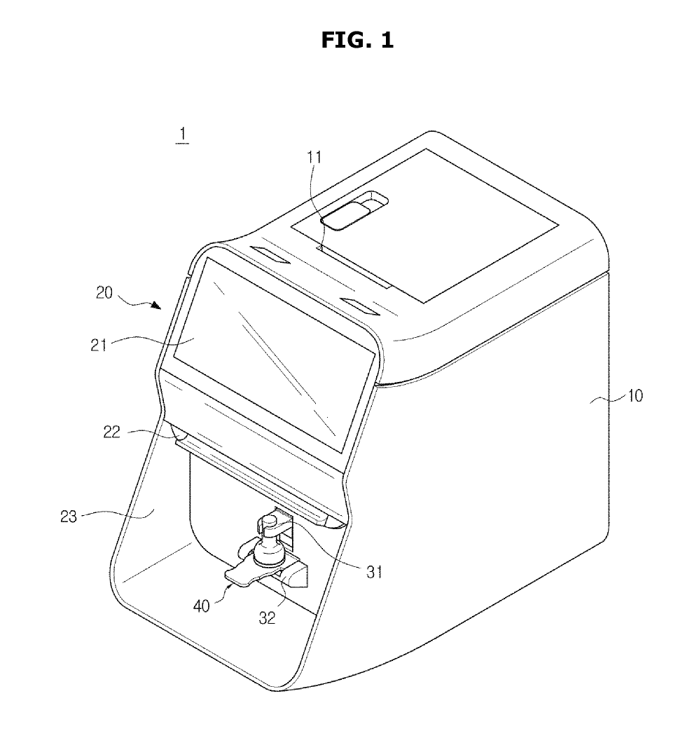

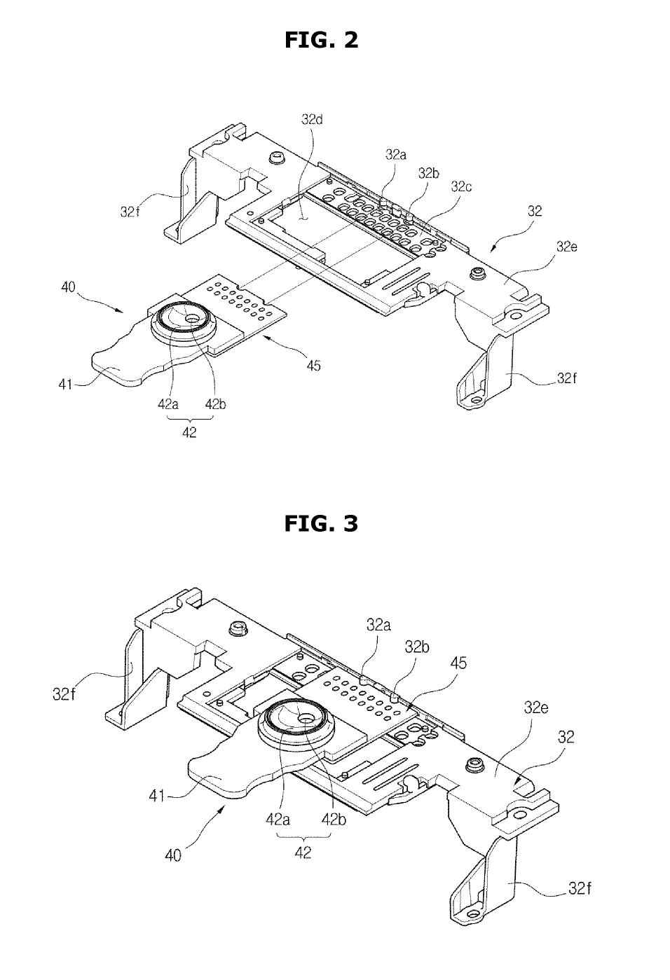

FIGS. 1 through 13, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably arranged specimen analysis apparatus.

Reference will now be made in detail to the embodiments of the present disclosure, examples of which are illustrated in the accompanying drawings, wherein like reference numerals refer to like elements throughout.

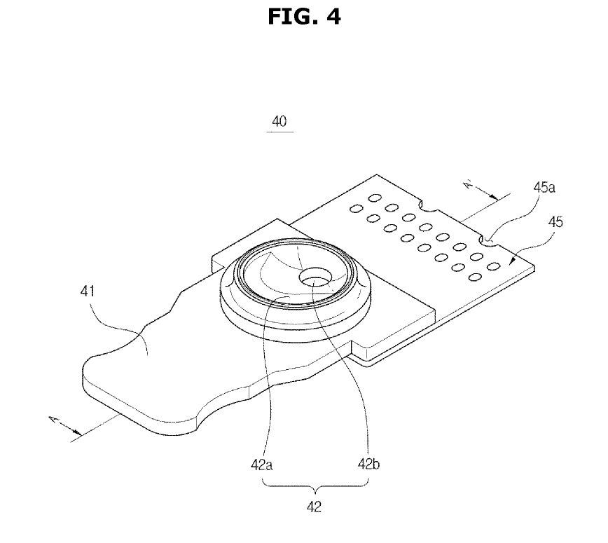

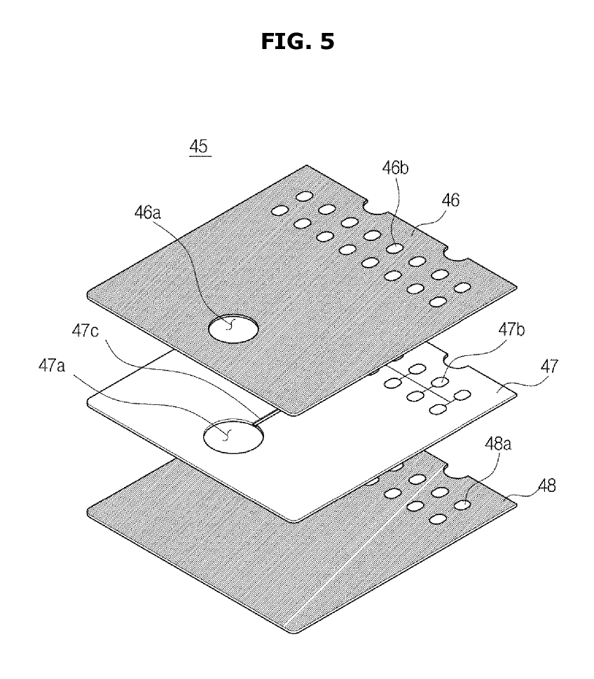

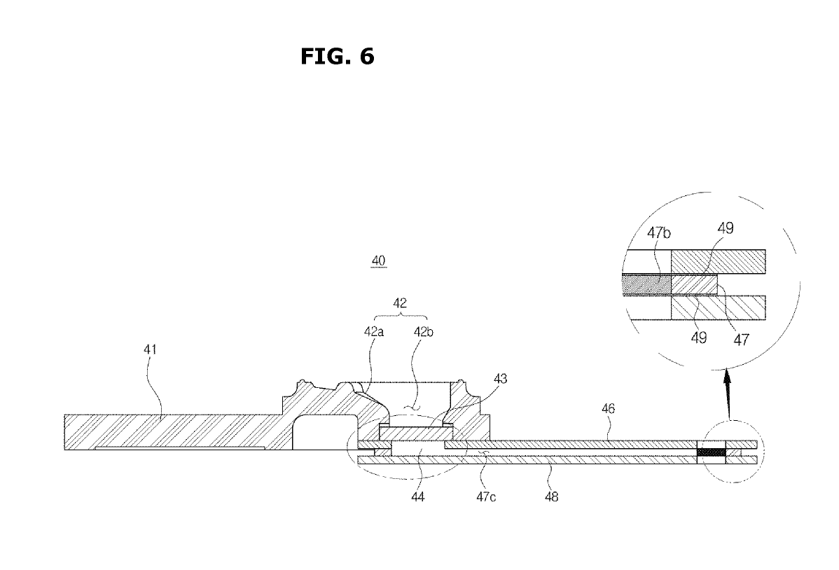

FIG. 1 illustrates a specimen analysis apparatus to which a cartridge is coupled according to an embodiment. FIG. 2 illustrates an exploded view of the cartridge and a mounting member of the specimen analysis apparatus to which the cartridge is coupled according to an embodiment. FIG. 3 illustrates a state in which the cartridge is coupled to the mounting member of the specimen analysis apparatus according to an embodiment. FIG. 4 illustrates a cartridge according to an embodiment. FIG. 5 is an exploded view illustrating a tester of the cartridge according to an embodiment. FIG. 6 illustrates a cross-sectional view of the tester of the cartridge illustrated in FIG. 4 taken along line AA′. Hereinafter, the drawings will be synthetically described to avoid repeated descriptions.

As illustrated in FIG. 1, a specimen analysis apparatus 1 to which a cartridge 40 is coupled according to an embodiment includes a housing 10 defining an appearance and a door module 20 disposed at the front of the housing 10.

The door module 20 may include a display 21, a door 22, and a door frame 23. The display 21 and the door 22 may be disposed at the front of the door frame 23. Although the display 21 may be located at an upper portion of the door 22 as illustrated in FIG. 1, the display 21 may also be located at any position enabling various information to be visually provided.

Meanwhile, the door 22 may be slidably installed. When the door 22 slides to be open, the door 22 may be disposed behind the display 21.

The display 21 may display various information regarding specimen analysis such as analysis information about a specimen and information about analysis state of the specimen. Besides, the display 21 may display various information about the specimen analysis apparatus 1 such as information about settings of the specimen analysis apparatus 1.

Meanwhile, the display 21, when implemented using a touchscreen, may receive various information, commands, and the like from a user. For example, the display 21 may display icons to input various control commands to control the specimen analysis apparatus 1 and normal pH ranges during analysis of the specimen,

According to an embodiment, the specimen analysis apparatus 1 may display a user interface configured to allow the user to easily recognize analysis results on the display 21. This will be described later.

Meanwhile, the door frame 23 may include a mounter member 32 on which the cartridge 40 accommodating various reagents is mounted. The user may perform an analysis operation by opening the door 22 by sliding the door 22 upward, mounting the cartridge 40 on the mounter member 32, and closing the door 22 by sliding the door 22 downward.

When the specimen is injected into the cartridge 40, reactions between the specimen and reagents take place in a tester 45. The specimen is introduced into the tester 45 when the mounter member 32 is inserted into the cartridge 40 and the cartridge 40 is pressed by a pressing member 31.

In addition, the specimen analysis apparatus 1 may further include an outputter 11 configured to output analysis results as a printout separately from the display 21. Thus, the user may output test results via the outputter 11 as well as identifying the test results via the display 21.

Meanwhile, referring to FIGS. 2 to 4, the cartridge 40 may be inserted into the mounter member 32 of the specimen analysis apparatus 1. The mounter member 32 may include a seating portion 32c on which the cartridge 40 is seated and supports 32f to support the mounter member 32 in the specimen analysis apparatus 1.

The supports 32f extend from both sides of a body 32e of the mounter member 32, and the seating portion 32c may be provided at the center of the body 32e. A slit 32d may be disposed behind the seating portion 32c to prevent errors of the tester 45 caused while testing the specimen sample.

The mounter member 32 may have contact portions 32a and 32b to contact the cartridge 40. The tester 45 of the cartridge 40 may include recesses 45a having a shape corresponding to those of the contact portions 32a and 32b. Thus, the recesses 45a may contact the contact portions 32a and 32b. In this case, two recesses 45a and two contact portions 32a and 32b corresponding thereto may be provided, without being limited thereto.

Meanwhile, the cartridge 40 includes a housing 41 defining an appearance of the cartridge 40 and the tester 45 in which reactions between the specimen and the reagents take place.

The housing 41 may support the cartridge 40 and have a shape to allow the user to grasp the cartridge 40. According to an embodiment, as illustrated in FIGS. 2 and 3, a portion grasped by the user may have a streamlined shape with protrusions. Thus, the user may stably grasp the cartridge 40. However, the shape of the housing 41 is not limited to that illustrated in the drawings and may also have various shapes without limitation.

Also, the cartridge 40 may have a specimen feeder 42 to receive the specimen. The specimen feeder 42 may include a feeding hole 42b through which the specimen sample is introduced into the tester 45 and a feeding assisting portion 42a to assist feeding of the specimen.

A specimen to be tested by the specimen analysis apparatus 1 is supplied into the specimen feeder 42, and examples of the specimen may include biological samples such as blood fluids including blood, tissue fluids, and lymph fluids, saliva, and urine or environmental samples for water-purity control or soil management, without being limited thereto. Meanwhile, the specimen sample may or may not be diluted, without being limited thereto.

Referring to the drawings, the feeding hole 42b may have a circular shape. However, the shape of the feeding hole 42b is not limited thereto, and various polygonal shapes may also be applied thereto. The user may drop the specimen into the specimen feeder 42 using a tool such as a pipette. The feeding assisting portion 42a is formed around the feeding hole 42b to be slanted downward toward to the feeding hole 42b, such that the specimen sample dropped around the feeding hole 42b flows into the feeding hole 42b. Particularly, when the user does not accurately drop the specimen sample into the feeding hole 42b and a part of the specimen sample is dropped around the feeding hole 42b, the specimen sample dropped around the feeding hole 42b may flow into the feeding hole 42b along the slanted surface of the feeding assisting portion 42a.

Also, the feeding assisting portion 42a may prevent contamination of the cartridge 40 by the inaccurately dropped specimen sample in addition to assisting the supply of the specimen sample. Particularly, even when the specimen sample does not accurately flow into the feeding hole 42b, the feeding assisting portion 42a formed around the feeding hole 42b prevents the inaccurately dropped specimen sample from flowing toward the tester 45 or the grasped portion. Thus, contamination of the cartridge 40 by the specimen sample may be prevented and contact between the specimen sample that might be harmful to the human body and the user may be prevented.

Although the specimen feeder 42 includes one feeding hole 42b in the drawings, the embodiment is not limited thereto, and a plurality of feeding holes may also be formed. When a plurality of feeding holes are provided, a plurality of different specimen samples may be simultaneously tested in one cartridge. Here, the plurality of different specimen samples may be the same type obtained from different sources, different types obtained from different sources, or the same type obtained from the same source in different states.

As described above, since the housing 41 has a shape suitable to perform specific functions and may contact the specimen sample, a chemically and biologically inactive material that is easily molded may be used to manufacture the housing 41.

For example, the housing 41 may be formed of various materials, for example, acryl such as polymethylmethacrylate (PMMA), polysiloxane such as polydimethylsiloxane (PDMS), polycarbonate (PC), polyethylene such as linear low density polyethylene (LLDPE), low density polyethylene (LDPE), medium density polyethylene (MDPE), and high density polyethylene (HDPE), plastic materials such as polyvinyl alcohol, very low density polyethylene (VLDPE), polypropylene (PP), acrylonitrile butadiene styrene (ABS), and cyclo olefin copolymer (COC), glass, mica, silica, and semiconductor wafer.

However, these materials are examples used to form the housing 41, and materials used to form the housing 41 are not limited thereto. The housing 41 according to an embodiment may also be formed using any chemically and biologically stable material with easy mechanical processability.

Meanwhile, the tester 45 may be bonded or joined to the cartridge 40. The specimen injected via the specimen feeder 42 is introduced into the tester 45 and tested as reactions between the specimen and the reagents take place. The tester 45 includes a plurality of accommodators 45b, and the reagents involved in reactions with the specimen may be accommodated in the accommodators 45b.

In this regard, the plurality of accommodators 45b may accommodate various types of reagents. For example, the accommodators 45b may accommodate a plurality of pH indicators with different properties. Accordingly, the specimen analysis apparatus 1 according to an embodiment may measure a pH of the specimen more accurately. This will be described later in more detail.

As another example, the accommodator 45b may accommodate reagents used to calculate a concentration of a target analyte in the specimen. In this case, since the reagents include coloring agents reacting with the target analyte, the specimen analysis apparatus 1 may detect optical characteristics via an optical sensor, or the like and calculate the concentration of the target analyte based on the detection results. Here, the optical sensor is a sensor emitting light and may include various types of known sensors such as an LED sensor or an infrared sensor, without limitation. Meanwhile, the specimen analysis apparatus 1 may detect optical characteristics of reactions between the pH indicators and the specimen using the optical sensor and deduce the pH measurement value based thereon.

Hereinafter, a structure of the tester 45 of the cartridge 40 will be described in more detail.

Referring to FIG. 5, the tester 45 of the cartridge 40 according to an embodiment may have a structure in which three plates 46, 47, and 48 are joined together. The three plates 46, 47, and 48 may be an upper plate 46, a middle plate 47, and a lower plate 48. The upper plate 46 and the lower plate 48 may be coated with a light blocking ink to protect the specimen sample flowing into the accommodator 45b from external light or prevent errors while measuring optical characteristics in the accommodator 45b. Each of the upper plate 46 and the lower plate 48 may have a thickness of 10 μm to 300 μm. The middle plate 47 may have a thickness of 50 μm to 300 μm.

A film used to form the upper plate 46 and the lower plate 48 of the tester 45 may be selected from a polyethylene film such as a very low density polyethylene (VLDPE) film, a linear low density polyethylene (LLDPE) film, a low density polyethylene (LDPE) film, a medium density polyethylene (MIDPE) film, and a high density polyethylene (HDPE) film, a polypropylene (PP) film, a poly(vinyl chloride) (PVC) film, a polyvinyl alcohol (PVA) film, a polystyrene (PS) film, and a polyethylene terephthalate (PET) film. However, these films are only examples, and any other chemically and biologically inactive films with mechanical processability may also be used to form the upper plate 46 and the lower plate 48 of the tester 45.

The middle plate 47 of the tester 45 may be a porous sheet different from the upper plate 46 and the lower plate 48. The porous sheet used to form the middle plate 47 may include at least one of cellulose acetate, Nylon 6.6, Nylon 6.10, and polyether sulfone. Due to the porous structure, the middle plate 47 may serve as vents, and thus the specimen sample may move in the tester 45 without having a separate driving source. Also, when the specimen sample is hydrophilic, the middle plate 47 may be coated with a hydrophobic solution to prevent the specimen sample from permeating into the inside of the middle plate 47.

The upper plate 46 have an inlet 46a through which the specimen sample is introduced and a region 46b corresponding to the accommodator 45b may be processed to be transparent. Also, a region 48a of the lower plate 48 corresponding to the accommodator 45b may be processed to be transparent. This processing is performed to measure optical density as an example of optical characteristics obtained by reactions taking place in the accommodator 45b.

The middle plate 47 may also include an inlet 47a through which the specimen sample is introduced, the inlet 46a of the upper plate 46 and the inlet 47a of the middle plate 47 overlap to form an inlet 44 of the tester 45. Also, the middle plate 47 may have a flow path 47c connecting the inlet 47a with a test portion 47b.

In the tester 45, various reactions for specimen analysis may take place. When blood is used as the specimen sample, a reagent, which reacts with a specific analyte of blood (particularly, plasma) and expresses a color or changes in color, may be accommodated in the accommodator 45b, and then a color expressed in the accommodator 45b may be optically detected and indicated in numeric values. Based on the numeric values, the presence of the analyte in the blood and a proportion of the analyte therein may be identified. Besides, when a pH indicator is accommodated in the accommodator 45b, the specimen analysis apparatus 1 according to an embodiment may deduce a pH measurement value by optically detecting a color expressed in the accommodator 45b and indicating the color in numeric values.

FIG. 6 is a cross-sectional view of the tester of the cartridge of FIG. 4 taken along line AA′.

In the cartridge 40, the tester 45 is adhered to an lower portion of the housing 41. More particularly, the tester 45 may be adhered to a portion of the specimen feeder 42 under the feeding hole 42b. The housing 41 may be adhered to the tester 45 using a pressure sensitive adhesive (PSA). The pressure sensitive adhesive may be adhered to an adherent within a short period of time at room temperature by a low pressure of about a finger pressure. When the pressure sensitive adhesive is peeled, cohesive failure is not caused and residues are not remained on the surface of the adherent. However, the housing 41 may be joined to the tester 45 by using not only the pressure sensitive adhesive, but also any other double-sided adhesives and grooves and protrusions coupled to each other.

The specimen sample introduced via the feeding hole 42b passes through a filter 43 as illustrated in FIG. 6 and enters the tester 45. The filter 43 may be inserted into the feeding hole 42b of the housing 41.

The filter 43 may include at least one porous membrane having a plurality of pores to filter materials having a predetermined size of greater and included in the specimen sample. For example, the filter 43 may include two layers of filters. According to an embodiment, a first filter may be formed of a glass fiber, an unwoven fabric, an absorbent filter, or the like, and a second filter may be formed of polycarbonate (PC), polyether sulfone (PES), polyethylene (PE), polysulfone (PS), or polyacrylsulfone (PASF).

When the filter 43 has a double-layered structure as described above, the specimen sample having passed through an upper filter may further be filtered by a lower filter. Also, when a large amount of particles greater than the pore size of the filter 43 is simultaneously introduced, tearing and damage of the filter 43 may be prevented. However, the embodiment is not limited thereto, and the filter 43 may include three or more layers. In this case, functions of filtering the specimen sample is further enhanced and stability of the filter 43 is further improved. Each filter 43 may be processed using an adhesive material (not shown) such as a double-sided adhesive.

The tester 45 may be provided with the inlet 44 through which the specimen sample flowing from the filter 43 is introduced, the flow path 47c through which the introduced specimen sample moves, and the accommodator 45b where reactions between the specimen sample and the reagents take place.

Meanwhile, the upper plate 46, the middle plate 47, and the lower plate 48 may be bonded by double-sided adhesive tapes 49. More particularly, the upper plate 46, the middle plate 47, and the lower plate 48 may be bonded by applying the double-sided adhesive tapes 49 to both an upper surface and a lower surface of the middle plate 47.

Hereinafter, a control block diagram of the specimen analysis apparatus 1 will be described.

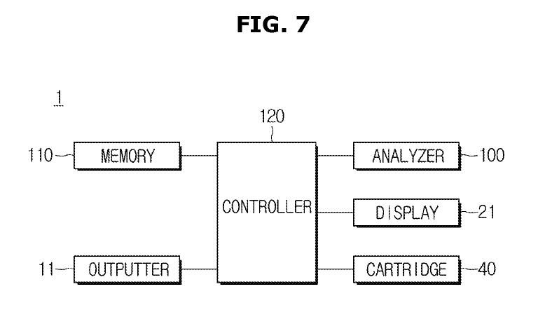

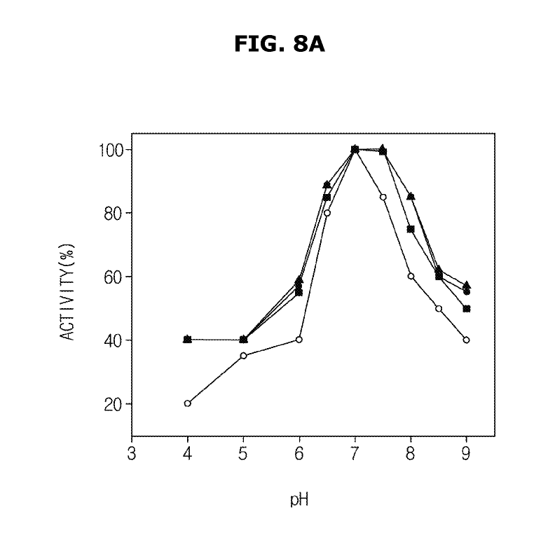

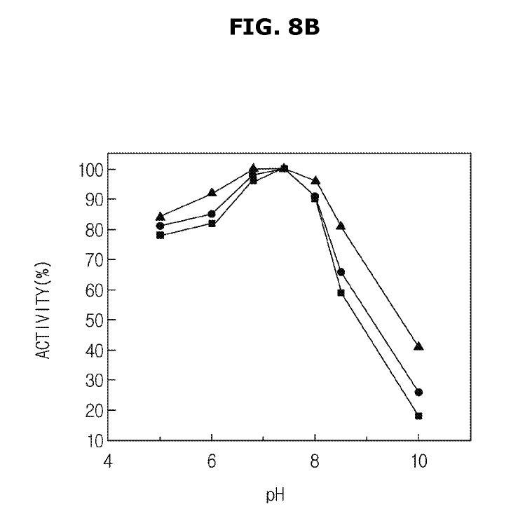

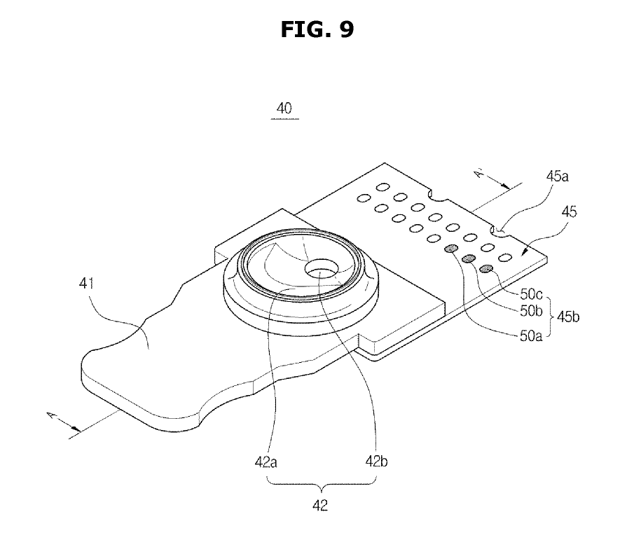

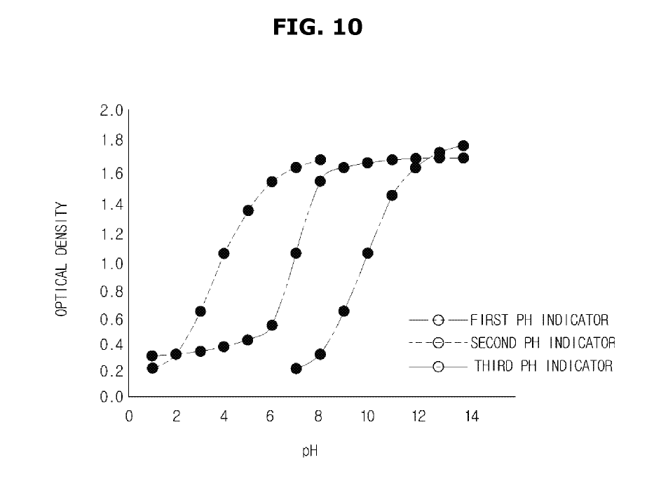

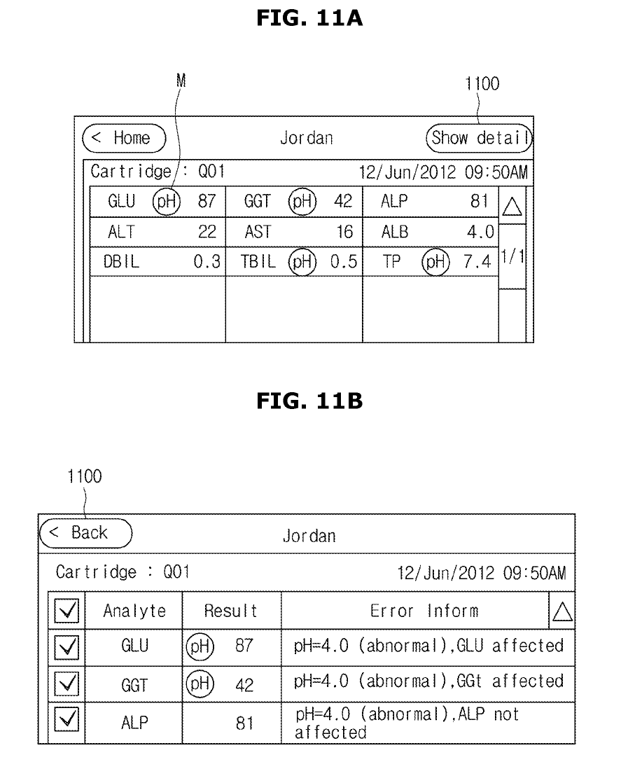

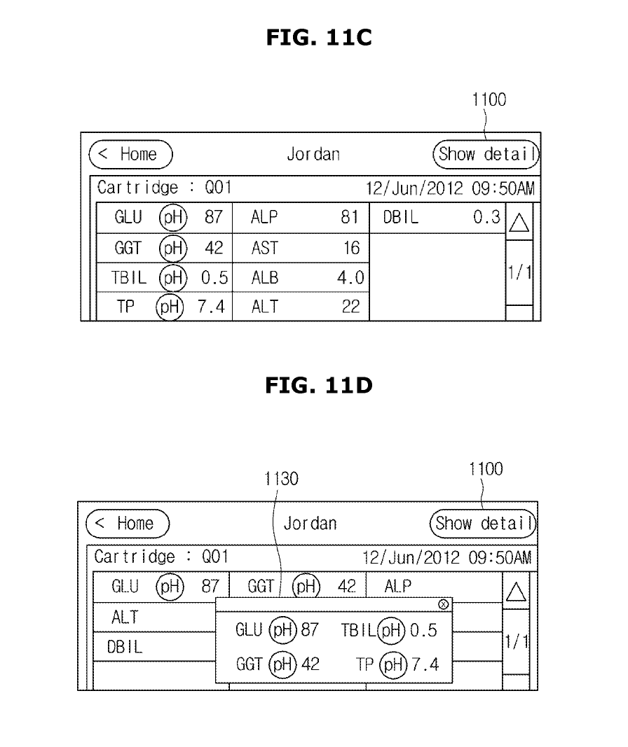

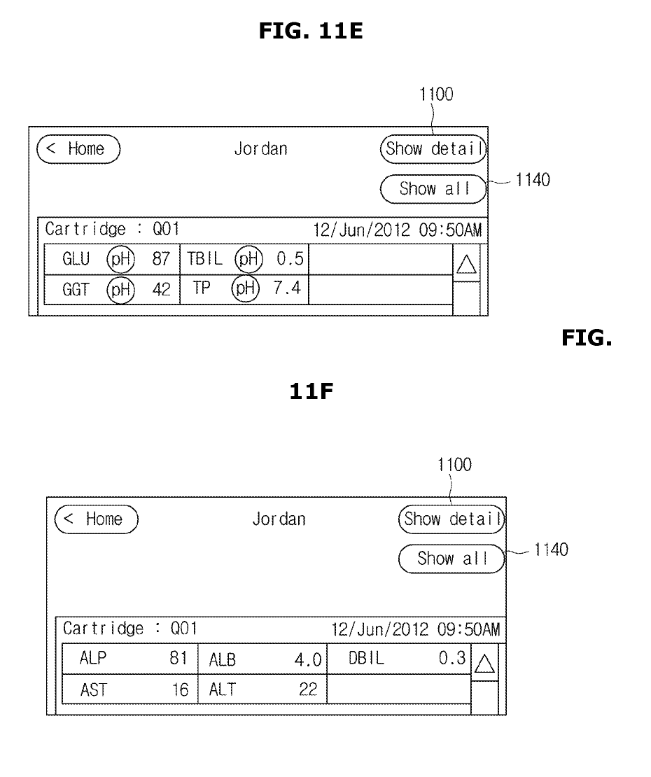

FIG. 7 illustrates a control block diagram of a specimen analysis apparatus according to an embodiment. FIGS. 8A and 8B illustrate graphs for describing activities of enzymes with respect to pH. FIG. 9 illustrates a cartridge accommodating reagents having different properties in a plurality of wells according to an embodiment. FIG. 10 illustrates a graph for describing color change ranges of pH indicators having different properties according to an embodiment. FIGS. 11A to 11F illustrate user interface screens displayed on a display according to an embodiment. Hereinafter, the drawings will be synthetically described to avoid repeated descriptions.

The specimen analysis apparatus 1 may further include an analyzer 100, a memory 110, and a controller 120 in addition to the aforementioned cartridge 40, display 21, and outputter 11.

Here, at least one of the analyzer 100, the memory 110, and the controller 120 may be integrated into a system on chip (SOC) embedded in the specimen analysis apparatus 1. However, since there is not only one SOC embedded in the specimen analysis apparatus, the embodiment is not limited to integration into one SOC. Meanwhile, the cartridge 40 and the display 21 are described above, and thus descriptions thereof will not be repeated.

The cartridge 40 accommodates various types of reagents enabling various analyses regarding the specimen. For example, the accommodator 45b of the cartridge 40 includes a plurality of wells spaced apart from each other to accommodate reagent including coloring agents reacting with the target analyte in the specimen and pH indicators measuring the pH of the specimen. Meanwhile, as described above, the specimen sample may not be diluted, without being limited thereto.

The target analyte refers to an analyte included in the specimen. For example, the target analyte refers to substances included in the specimen sample such as protein, enzyme, ion, and the like, without being limited thereto.

According to an embodiment, the user may identify a state of a subject by receiving analysis results about a concentration of the target analyte in the specimen via the specimen analysis apparatus 1. For example, if the target analyte is sodium ions, a concentration of sodium ions obtained by the analysis greater than a predetermined level may indicate that the subject may have symptoms of dehydration. Accordingly, the user may prescribe treatment for the subject based on the analysis results. That is, the user may accurately diagnose the subject by receiving analysis information about a desired target analyte among materials included in the specimen.

In this case, the analysis information about the target analyte in the specimen may be affected by the pH of the specimen.

The pH of the specimen sample may change in accordance with external environment. For example, when the specimen is exposed to air, the pH of the specimen sample changes. In this case, as the pH of the specimen sample changes, activities of the target analytes of the specimen sample change.

FIG. 8A illustrates a graph of changes in activity of urease with respect to the pH. FIG. 8B illustrates a graph of changes in activity of glucose with respect to the pH. Referring to FIGS. 8A and 8B, it may be confirmed that activities of the urease and glucose change in accordance with the pH. Thus, influence of the pH needs to be considered to obtain accurate analysis results about the target analyte. Also, the pH of the specimen needs to be accurately measured in order to consider the influence of the pH.

Thus, the analyzer 100 according to an embodiment may more accurately measure the pH of the specimen by using at least three pH indicators having different properties.

Meanwhile, the analyzer 100 may measure the pH value by measuring optical density obtained as a result of reactions between the specimen and the pH indicators using the optical sensor and indicating the optical density in numeric values. Here, the method of measuring the pH is not limited thereto, and various known methods may also be used.

For example, the accommodator 45b of the cartridge 40 may include a plurality of wells as illustrated in FIG. 9. In this case, a first well 50a, a second well 50b, and a third well 50c may accommodate pH indicators having different properties. Also, a reagent involved in reactions with a target analyte to be analyzed may be accommodated in another well, and the embodiment is not limited thereto.

The pH indicator is a reagent used to measure the pH, and the accommodator 45b may accommodate at least three pH indicators having different color change ranges, i.e., different sensitive regions. In this case, the pH indicator may be implemented using a liquid, a solidified gel, or a thin film, without being limited thereto.

According to an embodiment, a first pH indicator accommodated in the first well 50a may be a reagent having a color change range from an acidic pH to a basic pH. A second pH indicator accommodated in the second well 50b may be a reagent having a color change range from an acidic pH to neutral. A third pH indicator accommodated in the third well 50c may be a reagent having a color change ranged from neutral to a basic pH.

FIG. 10 illustrates a graph of optical density of the pH indicators with respect to the pH according to an embodiment. Although it is confirmed that the first pH indicator is sensitive in all regions, changes in optical density are not enough to accurately measure the pH value in other regions except for a neutral region as illustrated in FIG. 10. Also, despite high sensitivity under acidic conditions, the second pH indicator is insensitive under basic conditions. In addition, despite high sensitivity under basic conditions, the third pH indicator is insensitive under acidic conditions.

Thus, the pH value may be accurately measured by judging whether the pH of the specimen is acidic or basic, and then measuring the pH value using a pH indicator sensitive under acidic conditions or basic conditions, i.e., a pH indicator exhibiting a greater change in optical density under acidic conditions or basic conditions, selected based on results of the judgement.

Accordingly, the analyzer 100 according to an embodiment may measure the pH value by a stepwise process using a plurality of pH indicators having different color change ranges, i.e., different sensitive regions.

For example, the analyzer 100 according to an embodiment may judge whether the specimen is acidic or basic based on a pH value measured using one pH indicator having the widest color change range among the plurality of pH indicators accommodated in the accommodator 45b, and then select one pH measurement value obtained using another pH indicator selected upon results of the judgement as a pH measurement value.

Thus, the specimen analysis apparatus 1 according to an embodiment may analyze the specimen sample more accurately by more accurately deducing the pH measurement value, determining whether the pH measurement value is within a normal range, and providing determination results. Hereinafter, the method of measuring the pH value by the analyzer 100 will be described in more detail.

The analyzer 100 may judge the pH region of the specimen by using the pH indicator having the widest color change range among the pH indicators accommodated in the accommodator 45b. As described above, the pH indicators may have different color change ranges, i.e., different reaction sensitivities with respect to the pH.

The accommodator 45b may accommodate at least three pH indicators. In this regard, the at least three pH indicators have different color change ranges, and other pH indicators may have the same or different color change ranges.

In this case, the analyzer 100 may determine a pH region of the specimen by using the pH indicator having the widest color change range among the at least three pH indicators accommodated in the accommodator 45b and having different properties. That is, the analyzer 100 may determine the pH region of the specimen via a pH value measured by using a pH indicator having the largest reaction region among the pH indicators accommodated in the accommodator 45b.

However, the pH indicator having the widest color change range has a lower sensitivity with respect to pH changes. For example, the first indicator exhibits less changes in optical density under the acidic and basic conditions as illustrated in FIG. 10.

Thus, the analyzer 100 according to an embodiment may determine a pH measurement value obtained by using a pH indicator having the highest sensitivity in the pH region of the specimen among the pH indicators accommodated in the accommodator 45b as the pH measurement value of the specimen. For example, the analyzer 100 may calculate the pH measurement value by measuring optical density from reactions between the pH indicators and the specimen and indicating the optical density in numeric values.

That is, the analyzer 100 according to an embodiment may approximately determine the pH region of the specimen, select a pH indicator capable of accurately obtaining the pH value in the pH region of the specimen among the pH indicators accommodated in the accommodator 45b, and determine a pH measurement value obtained using the selected pH indicator as the pH measurement value of the specimen. Thus, the analyzer 100 according to an embodiment may more accurately measure the pH by obtaining the pH measurement value using a stepwise process.

Also, the analyzer 100 may judge whether the pH measurement value is normal or abnormal. For example, the analyzer 100 may judge whether the pH measurement value is out of a normal range by comparing data about the normal pH range of the specimen stored in the memory 110 with the actually measured pH value.

As described above, the pH of the specimen sample changes in accordance with external environment. As the pH of the specimen sample changes, activities of the target analytes change. Thus, if the pH measurement value is out of the normal range, analysis results of the target analyte is affected thereby.

Accordingly, in order to provide more accurate analysis results about the target analyte, the analyzer 100 according to an embodiment may further provide a result of judging whether the pH measurement value is within the normal range, and thus the user may use the result as a reference while diagnosing a patent.

In addition, when the pH measurement value is out of the normal range, the analyzer 100 according to an embodiment may correct the analysis results about the target analyte by reflecting the degree of influence of the pH value thereto. The correction may be performed by using various methods well known in the art, without limitation. Meanwhile, the analyzer 100 may also measure the pH value of the specimen using various known other methods, without being limited thereto.

In addition, the analyzer 100 may obtain analysis information about the target analyte in the specimen using the reagents accommodated in the accommodator 45b. For example, the accommodator 45b may include reagents including coloring agents reacting with the target analyte to be analyzed. Thus, the analyzer 100 may obtain a concentration of the target analyte. According to an embodiment, the analyzer 100 may deduce the concentration of the target analyte by detecting reactions between the reagents and the specimen via the optical sensor, and indicate the reaction results in numeric values.

The memory 110 stores data about optical density changes with respect to concentrations of the target analyte, which will be described later. Accordingly, the analyzer 100 may deduce the concentration corresponding to the optical density using data stored in the memory 110.

As described above, the specimen analysis apparatus 1 may include the memory 110.

In this regard, the memory 110 may be implemented using at least one storage medium selected from a flash memory type, a hard disk type, a multimedia card micro type, a card-type memory (e.g., secure digital (SD) card or an extreme digital (XD) card), a random access memory (RAM), a static random access memory (SRAM), a read only memory (ROM), an electrically erasable programmable read-only memory (EEPROM), a programmable read only memory (PROM), a magnetic memory, a magnetic disc, and an optical disc. However, the memory 110 is not limited thereto and may also be implemented using any other types of memory.

The memory 110 may store data about optical density changes of the target analytes in accordance with the pH changes. In this case, optical density changes of the target analytes in accordance with pH changes may be the same or different depending on the types of the reagents. Thus, the memory 110 may store data regarding optical density changes in accordance with the pH changes on the basis of the reagent and the target analyte.

Also, the memory 110 may store data of a normal pH range of the specimen. Particularly, normal pH ranges of the target analytes in the specimen stored in the memory 110 may be different. In this case, the memory 110 may store data about the normal pH ranges of the target analytes in the specimen.

The normal pH ranges of the target analytes in the specimen may be set by the user or by a designer of the specimen analysis apparatus 1. For example, the normal pH ranges may vary according to race and age of the subject. Also, the normal pH ranges of the target analytes in the specimen may vary according to properties of the pH indicators.

Thus, the user may directly set the normal pH ranges by considering characteristics of the subject from which the specimen is extracted or properties of the pH indicators. According to an embodiment, when the display 21 is implemented using a touchscreen, the user may set the normal pH ranges by touching or clicking the display 21. The controller 120 may allow the user to easily input various settings by displaying a user interface to input the normal pH ranges, and the like, which will be described later, on the display 21. The user interface will be described later in more detail.

Meanwhile, data stored in the memory 110 may be updated. For example, data about the normal pH range stored in the memory 110 may be updated by a wired or wireless communication network, without being limited thereto.

Also, the data stored in the memory 110 may be automatically updated periodically at predetermined intervals. For example, the data about the normal pH ranges stored in the memory 110 may be updated via a wireless or wired communication network periodically at update intervals preset by the user.

Meanwhile, the memory 110 may store data about a user interface. Here, the user interface refers to an environment configured to allow the user to easily input various setting commands and control commands about the specimen analysis apparatus 1, easily control programs stored in the memory 110, and easily recognize various information such as analysis results through the specimen analysis apparatus 1.

For example, the user interface may be a graphic user interface realizing a screen displayed on the display 21 as a graph to facilitate communications of various information, commands, and the like between the user and the specimen analysis apparatus 1.

In this case, methods of providing various information via the user interface, methods of displaying and arranging icons to receive various setting commands, control commands, and the like, may be implemented using algorithms or programs and stored in the memory 110. Thus, the controller 120 may create the user interface using data stored in the memory 110 and display the user interface on the display 21. Alternatively, the aforementioned algorithms, programs, and the like may be stored in external devices. Accordingly, the controller 120 may receive data about the user interface deduced by the external devices using the algorithms and programs via a communication network and display the data on the display 21. However, the embodiment is not limited thereto.

Meanwhile, the specimen analysis apparatus 1 may include the controller 120. The controller 120 may be implemented using a processor such as a micro controller (MCU).

The controller 120 may control elements of the specimen analysis apparatus 1 via control signals. For example, when a command to output analysis results is received from the user, the controller 120 may control the outputter 11 to output the analysis results via a control signal.

As another example, the controller 120 may display analysis information about the specimen by controlling the display 21 via a control signal. In this case, the controller 120 may control the user interface configured to provide analysis information about the target analyte in the specimen and pH analysis information about the specimen to be displayed on the display 21.

As described above, the memory 110 may store data about the user interface. Thus, the controller 120 may detect whether the pH affects the analysis information of the specimen using data stored in the memory 110 when the user performs a diagnosis.

For example, the controller 120 may enable the user to judge whether analysis information about the target analyte in the specimen is affected by the pH by realizing a user interface configured to provide at least one of analysis information about the target analyte in the specimen, the pH measurement value, and information about whether the pH measurement value is within the normal pH range, and displaying the user interface on the display 21.

The controller 120 may create a user interface suitable for the user or suitable for a situation by using data stored in the memory 110 and display the user interface on the display 21.

FIG. 11A illustrates a screen of the display 21 according to an embodiment displaying a user interface on the display 21. The user interface may display various information, and icons may also be arranged thereon to receive various commands from the user.

A name or title of the subject, e.g., ‘Jordan’, may be displayed on the user interface, and test time, e.g., ‘12/Jun/2012 09:50 AM’, may be displayed thereon as illustrated in FIG. 11A.

In this case, the user interface may display analysis information such as a concentration of the target analyte in the specimen such as GLU, GGT, and ALP. Upon determination that the pH measurement value of the target analyte is out of the normal pH range, the user interface may display a mark M beside the analysis information as illustrated in FIG. 11A.

Meanwhile, when the user clicks or touches a “show detail” icon 1100, the controller 120 may change the user interface into another user interface to provide more detailed analysis information about the pH value and display the detailed information on the display 21 as illustrated in FIG. 11B.

Referring to FIG. 11B, the controller 120 may display the user interface configured to provide the pH measurement value of the target analyte, whether the measurement value is within the normal pH range, and whether the pH measurement value affected analysis of the target analyte, on the display 21.

More particularly, the controller 120 may display that the pH measurement value is 4.0 (pH=4.0), the pH measurement value is out of the normal pH range (abnormal), and the pH affected the concentration analysis of GLU (GLU affected) simultaneously displaying that the concentration of the target analyte GLU is 87 as illustrated in FIG. 11B. However, the aforementioned information is not limited to these terms illustrated in FIG. 11B, various linguistic expressions and other expression methods may also be used to provide the information. Meanwhile, when the user clicks or touches a “back” icon 1110, the controller 120 may convert the user interface into that illustrated in FIG. 11A and display the converted user interface on the display 21.

However, the user interface is not limited thereto. For example, the controller 120 may also display a user interface that classifies the target analytes into target analytes having abnormal pH measurement values and target analytes having normal pH measurement values and displays the classified target analytes on the display 21 as illustrated in FIG. 11C.

According to another embodiment, the controller 120 may realize a user interface that displays a list of target analytes determined to have abnormal pH measurement values as a popup message 1130. That is, the user interface may be implemented using various methods to provide analysis information about the target analytes, analysis information about the pH values of the target analytes, and the like, without limitation.

According to another embodiment, the controller 120 may also realize a user interface including a menu, icon, or the like used to set whether the pH measurement value is within the normal pH range. Accordingly, the controller 120 may or may not display the pH mark in accordance with the user's settings. That is, the controller 120 may increase the degree of freedom of the user to select methods of providing analysis results via the specimen analysis apparatus 1, and thus the user may diagnose the specimen more freely.

According to another embodiment, the controller 120 may realize a user interface configured to provide analysis information only about target analytes having normal pH measurement values or only about target analytes having abnormal pH measurement values. According to an embodiment, when the user sets the user interface to provide analysis information only about the target analytes having the abnormal pH measurement values, the controller 120 may display a user interface illustrated in FIG. 11E on the display 21. In this case, by clicking or touching a “show all” icon 1140, the controller 120 may convert the screen into that illustrated in FIG. 11A providing analysis information about all target analytes.

According to another embodiment, when the user sets the user interface to provide analysis information only about the target analytes having the normal pH measurement values, the controller 120 may display a user interface illustrated in FIG. 11F on the display 21. That is, the specimen analysis apparatus 1 according to an embodiment may realize the user interface in accordance with the user's setting, without limitation.

Meanwhile, the controller 120 and the memory 110 may be implemented using single chips, respectively. However, the embodiment is not limited thereto, and the controller 120 and the memory 110 may be implemented using a single chip.

Hereinafter, an operation flow of measuring the pH of the specimen using the plurality of pH indicators having different properties will be described separately from an operation flow of displaying the user interface configured to provide analysis information about the specimen and analysis information about the pH of the specimen on the display.

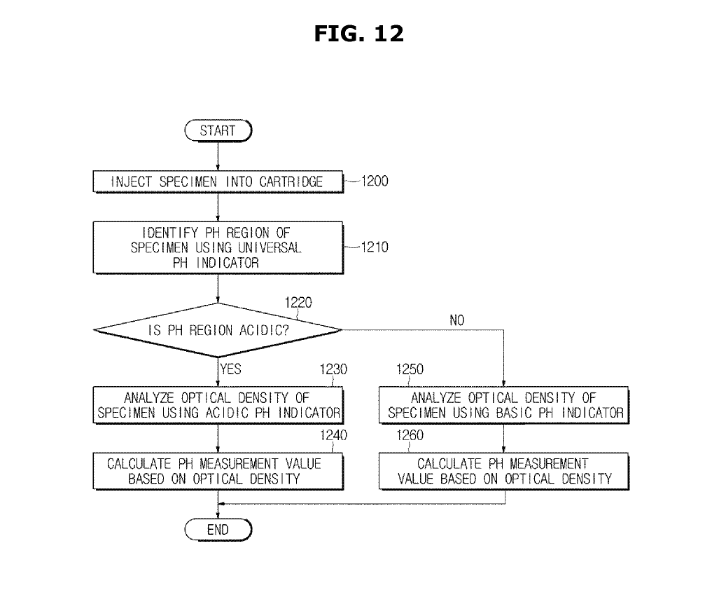

FIG. 12 illustrates a flowchart for describing a process of measuring the pH of the specimen using the plurality of pH indicators by the specimen analysis apparatus according to an embodiment.

A specimen may be injected into the cartridge coupled to the specimen analysis apparatus and a reagent reacting with the specimen may be injected thereinto (1200). For example, referring to FIG. 1, when the cartridge 40 is inserted into the mounter member 32 of the specimen analysis apparatus 1, and the pressing member 31 presses the cartridge 40, the specimen injected into the cartridge 40 is introduced into the tester 45 in which various reagents are accommodated.

Thus, the specimen analysis apparatus may deduce various analysis information about the specimen from reaction between the specimen and the reagents. In this case, the specimen analysis apparatus may detect optical density, as an example of optical characteristics of reactions between the specimen and the reagents, by using the optical sensor. Then, the specimen analysis apparatus may measure the concentration of the target analyte in the specimen, the pH of the target analyte, and the like by indicating the optical density in numeric values.

According to an embodiment, the specimen analysis apparatus may measure the pH values of the specimen or the target analyte in the specimen by using a universal pH indicator. In this case, although the universal pH indicator has a wide color change range, i.e., a wide pH measuring range, it is difficult to accurately measure the pH value thereby.

Accordingly, after the specimen analysis apparatus identifies the pH range of the specimen using the universal pH indicator (1210), the pH measurement value may be determined depending on whether the specimen is acidic or basic (1220).

Here, data about the pH values may be stored in the memory of the specimen analysis apparatus. For example, data about the pH values of the optical density may be stored in the memory on the basis of the pH indicator or on the basis of the specimen. Accordingly, the specimen analysis apparatus may identify the pH range of the specimen or the target analyte in the specimen by calculating the pH measurement value corresponding to the optical density using data stored in the memory.

When the pH of the specimen is in the acidic range, the specimen analysis apparatus may analyze the optical density of the specimen using an acidic pH indicator (1230). Thus, the specimen analysis apparatus may calculate the pH measurement value of the specimen by calculating the pH value corresponding to the analyzed optical density in numeric values using data stored in the memory (1240).

Then, the specimen analysis apparatus may determine the pH measurement value obtained using the acidic pH indicator as the pH measurement value of the specimen of the target analyte in the specimen and judge whether the pH value of the specimen is within the normal range based thereon. Accordingly, the specimen analysis apparatus may perform diagnosis more accurately by providing not only analysis information about the specimen such as the concentration of the target analyte but also analysis information about the pH value affecting the analysis information about the specimen.

Meanwhile, when the pH of the specimen is in the basic range, the specimen analysis apparatus may analyze the optical density of the specimen using a basic pH indicator (1250). Then, the specimen analysis apparatus may calculate the pH measurement value based on the optical density (1260). Here, the calculation is performed in the same manner as described above, and descriptions thereof will not be repeated.

The specimen analysis apparatus may determine the pH measurement value obtained using the basic pH indicator as the pH measurement value of the specimen and judge whether the pH value of the specimen is within the normal range based thereon. Accordingly, the specimen analysis apparatus may perform diagnosis more accurately by providing not only analysis information about the specimen such as the concentration of the target analyte but also analysis information about the pH affecting the analysis information about the specimen.

Furthermore, when the pH measurement value of the specimen is abnormal, the specimen analysis apparatus may correct the analysis results of the specimen by reflecting the abnormal data thereto. Correction may be performed using various methods well known in the art, without limitation.

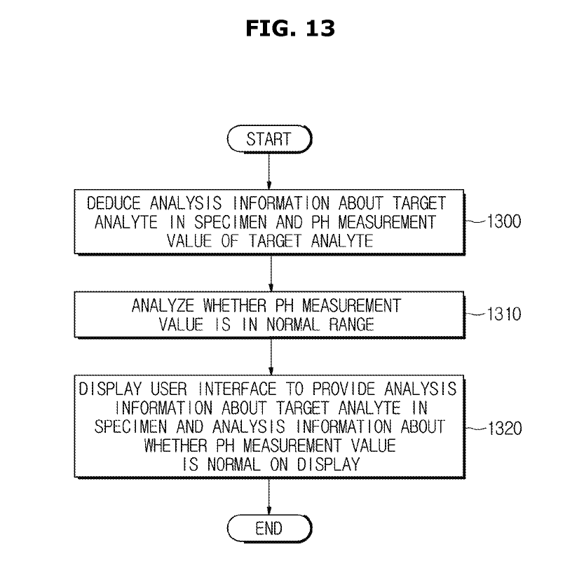

FIG. 13 illustrates a flowchart for describing a process of displaying a user interface configured to provide analysis information about the target analyte in the specimen together with analysis information about the pH value of the target analyte on the display by the specimen analysis apparatus.

The specimen analysis apparatus may extract analysis results by analyzing the specimen. In this case, the specimen analysis apparatus may extract analysis information about the specimen and analysis information about the pH of the specimen using various types of reagents (1300).

In this regard, the analysis information about the specimen includes information about the presence of the target analyte in the specimen and the concentration of the target analyte. Also, the analysis information about the pH of the specimen includes information about the pH measurement values of the specimen or the target analyte in the specimen. For example, the specimen analysis apparatus may deduce not only the analysis results about the concentration of the target analyte in the specimen but also the analysis results about the pH of the specimen by analyzing optical density of the specimen using various types of reagents including coloring agents.

Meanwhile, when the pH of the specimen is analyzed, the specimen analysis apparatus may use at least three pH indicators having different properties as described above. However, analysis information about the pH included in a user interface, which will be described later, is not limited to that obtained using the aforementioned analysis method but may also be obtained using various other known methods, without being limited thereto.

The specimen analysis apparatus may determine whether the measured pH value is within the normal pH range (1310). In this case, information about the normal pH range may be set on the basis of the specimen or the target analyte in the specimen and stored in the memory of the specimen analysis apparatus.

The normal pH range may be directly set by the user or preset and then stored in the memory. For example, the normal pH range may vary according to age, gender, and race of the subject. Thus, the specimen analysis apparatus according to an embodiment may inhibit misjudgment and accurately correct the analysis information about the target analyte by allowing the user to directly set the normal pH range.

The specimen analysis apparatus may display a user interface configured to provide analysis information about the specimen and analysis information about whether the pH measurement value is within the normal range on the display (1320). Thus, the user may easily recognize the analysis results about the specimen.

The specimen analysis apparatus according to an embodiment may realize the user interface using various methods as illustrated in FIGS. 11A to 11F and display the user interface on the display. Thus, the user may recognize whether the pH value is normal in addition to the analysis information about the specimen via the display, thereby accurately diagnosing the specimen.

Although the exemplary embodiments of the present disclosure have been provided for illustrative purposes, those skilled in the art will appreciate that various modifications are possible, without departing from the scope and spirit of the disclosure as disclosed in the accompanying claims.

The terms used in the present specification are merely used to describe particular embodiments, and are not intended to limit the present disclosure. An expression used in the singular encompasses the expression of the plural, unless it has a clearly different meaning in the context. In the present specification, it is to be understood that the terms such as “including” or “having,” etc., are intended to indicate the existence of the features, numbers, operations, components, parts, or combinations thereof disclosed in the specification, and are not intended to preclude the possibility that one or more other features, numbers, operations, components, parts, or combinations thereof may exist or may be added.

It will be understood that, although the terms “first”, “second”, etc., may be used herein to describe various elements, these elements should not be limited by these terms. The above terms are used only to distinguish one component from another. For example, a first component discussed below could be termed a second component, and similarly, a second component may be termed a first component without departing from the teachings of this disclosure. As used herein, the term “and/or” includes any and all combinations of one or more of the associated listed items.

In addition, the terms “unit”, “device,” “block”, “member”, and “module” used herein refer to a unit which can be embodied as software stored in a memory, hardware such as field-programmable gate array (FPGA) or application specific integrated circuit (ASIC), or a combination thereof, for processing at least one function and performing an operation. However, the terms “unit”, “device,” “block”, “member”, and “module” are not limited to software or hardware. The “unit”, “device,” “block”, “member”, and “module” may be stored in a storage medium and implemented by one or more processors.

Although the present disclosure has been described with an exemplary embodiment, various changes and modifications may be suggested to one skilled in the art. It is intended that the present disclosure encompass such changes and modifications as fall within the scope of the appended claims.

What is claimed is:

1. A specimen analysis apparatus comprising:
a cartridge configured to accommodate at least three pH indicators having different properties; and
an analyzer configured to:
determine whether a specimen is acidic or basic using a pH indicators from the at least three pH indicators; and
determine a pH measurement value using another pH indicator from the at least three pH indicators selected based on the determination that the specimen is acidic or basic.

2. The specimen analysis apparatus according to claim 1, wherein the at least three pH indicators have different color change ranges.

3. The specimen analysis apparatus according to claim 1, wherein the pH indicator used to determine whether the specimen is acidic or basic has a widest color change range among the at least three pH indicators, and the another pH indicator used to determine the pH measurement value is selected from at least two pH indicators having different color change ranges based on the determination that the specimen is acidic or basic.

4. The specimen analysis apparatus according to claim 1, further comprising a controller configured to:
generate a user interface configured to provide analysis information about a target analyte in the specimen and information about whether a pH measurement value of the target analyte in the specimen is in a normal range; and
display the user interface on a display.

5. The specimen analysis apparatus according to claim 1, wherein the at least three pH indicators comprises a universal pH indicator, a basic pH indicator, and an acidic pH indicator.

6. The specimen analysis apparatus according to claim 1, wherein the analyzer is configured to measure a pH value using optical density.

7. The specimen analysis apparatus according to claim 1, wherein the at least three pH indicators are composed of a solidified gel or a thin film.

8. A specimen analysis apparatus comprising:
an analyzer configured to:
determine analysis information about a specimen and a pH measurement value of the specimen; and
analyze whether the pH measurement value is within a normal range; and
a display configured to display a user interface configured to provide the analysis information about the specimen and analysis information about whether the pH measurement value is within the normal range.

9. The specimen analysis apparatus according to claim 8, wherein the display is configured to display the user interface through which a user sets normal ranges of target analytes in the specimen.

10. The specimen analysis apparatus according to claim 9, wherein the display is configured to display the user interface configured to set analysis information about whether the pH measure value is within the normal range.

11. The specimen analysis apparatus according to claim 8, wherein the analyzer is configured to:
determine whether the specimen is acidic or basic using a pH indicator having a widest color change range among at least three pH indicators having different color change ranges and accommodated in a cartridge; and
determine a pH measurement value using another pH indicator selected based on results of the determination whether the specimen is acidic or basic.

12. The specimen analysis apparatus according to claim 8, further comprising a memory configured to store data about normal ranges of target analytes in the specimen.

13. A method of analyzing a specimen using a specimen analysis apparatus accommodating at least three pH indicators having different properties, the method comprising:
determining whether a specimen is acidic or basic using a pH indicators from the at least three pH indicators; and
determining a pH measurement value obtained using another pH indicator from the at least three pH indicators selected based on results of the determination whether the specimen is acidic or basic.

14. The method according to claim 13, wherein the at least three pH indicators having different color change ranges.

15. The method according to claim 13, wherein the pH indicator having a widest color change range among the at least three pH indicators is used to determine whether the specimen is acidic or basic, and another pH indicator selected from at least two pH indicators having different color change ranges based on whether the specimen is acidic or basic is used to determine the pH measurement value of the specimen.

16. The method according to claim 13, further comprising:
generating a user interface configured to provide analysis information about a target analyte in the specimen and information about whether a pH measurement value of the target analyte in the specimen is in a normal range; and
displaying the user interface on a display.

17. The method according to claim 16, further comprising storing data about the normal ranges of the target analytes in the specimen.

18. The method according to claim 13, wherein the at least three pH indicators comprises a universal pH indicator, a basic pH indicator, and an acidic pH indicator.

19. The method according to claim 13, wherein the pH measurement value is measured using optical density.

20. The method according to claim 13, wherein the at least three pH indicators are composed of a solidified gel or a thin film.
